(19) United States
(12) Patent Application Publication
(10) Pub. No.: US 2006/0105022 A1
(43) Pub. Date: May 18, 2006

(54) Process for preparing crosslinked hyaluronic acid gel

(75) Inventors: Yoshihiro Yokokawa, Yokohama-shi (JP); Takashi Oka, Yokohama-shi (JP); Yuichiro Mori, Yokohama-shi (JP); Norio Ueno, Yokohama-shi (JP)

Correspondence Address: RANKIN, HILL, PORTER & CLARK, LLP, 925 EUCLID AVENUE, SUITE 700, CLEVELAND, OH, 44115-1405, US

(73) Assignee: Shiseido Co., Ltd., Chuo-ku (JP)

(21) Appl. No.: 11/272,914

(22) Filed: Nov. 14, 2005

Related U.S. Application Data
(60) Provisional application No. 60/627,886, filed on Nov. 15, 2004.

(51) Int. Cl. A61K 31/728; C08B 37/00; A61K 47/00

(52) U.S. Cl. 424/439; 514/54; 536/53

(57) ABSTRACT

[PROBLEMS] A novel process which can simply prepare a crosslinked hyaluronic acid gel having a small crosslinking agent content and exhibiting excellent viscoelasticity is provided. [MEANS TO SOLVE] A method of producing cross-linking hyaluronic acid gel, comprising stirring and mixing a mixture containing 20 W/V % or more of hyaluronic acid, a crosslinking agent and water under acidic or alkaline condition.

### FIELD OF THE ART

The present invention relates to a method of producing cross-linking hyaluronic acid gel, more particularly, a process for more simply preparing a crosslinked hyaluronic acid gel having a low crosslinking rate and excellent viscoelasticity.

### BACKGOUND ART

A crosslinked hyaluronic acid gel obtained by crosslinking hyaluronic acid is excellent in biocompatibility and, at the same time, also has such the biodegradability that it is progressively degraded in a living body with time, and is finally extinguished. Previously, utilizing this nature of a crosslinked hyaluronic acid gel, study and development regarding application to adhesion preventing agents, bone repairing agents, drug sustained release compositions, and tissue increasing substances have been extensively performed. Among them, as a representative example of application to tissue increasing substances, antiwrincle injections in the field of aesthetic plastic surgery.

A crosslinked hyaluronic acid gel is generally prepared by stirring and mixing hyaluronic acid and a crosslinking agent in an aqueous solution, to chemically combine between hyaluronic acid polymer chains by a crosslinking agent. Herein, when such the crosslinked hyaluronic acid gel is administered to a living body, it is feared that a gel is degraded in a living body, a remaining crosslinking agent component is recognized as a foreign matter to a living body, and this adversely influences such as causing an inflammation reaction. For this reason, when maintenance of biocompatibility is intended, it is desired that a crosslinked hyaluronic acid gel is prepared at as a low crosslinking rate as possible.

However, in the previous general crosslinking method, when an amount of a crosslinking agent to be added is reduced, viscoelasticity of the resulting crosslinked hyaluronic acid gel is reduced, and the gel becomes soft and, for example, when it is used as an antiwrinkle injection, a constant volume cannot be maintained at an injected site. In addition, when a crosslinked hyaluronic acid gel is used as a drug sustained release preparation, in order to maintain proper effect of a drug for a constant term, it is required that a drug resides in a living body for a long term, and a denser high viscoelastic crosslinked gel is required. However, when an amount of a crosslinking agent is reduced, it is difficult to obtain such the high viscoelastic crosslinked gel. Like this, it was a very difficult object to prepare a crosslinked hyaluronic acid gel having both of a low crosslinking rate and excellent viscoelasticity.

With respect to this object, Patent document 1 reports a process for preparing a crosslinked hyaluronic acid gel having a relatively low crosslinking rate and excellent viscoelasticity. However, this process, specifically, is via a two-stage crosslinking reaction step as follows: hyaluronic acid and a crosslinking agent are mixed in an aqueous solution to initiate a crosslinking reaction and, by adding water before occurrence of gelling to dilute a mixed solution, progression of a crosslinking reaction is prevented once, and a crosslinking reaction is progressed again by volatilizing water from this mixed solution. Therefore, there is a problem that operation is very troublesome, control of a reaction is difficult, and the process is not suitable for mass production.

[PATENT DOCUMENT 1] Japanese Patent No. 3094074

### PROBLEM TO BE SOLVED BY THE INVENTION

The present invention was done in view of the aforementioned problem of the prior art, and an object thereof is to provide a novel process for simply preparing a crosslinked hyaluronic acid gel having a low crosslinking rate and exhibiting excellent viscoelasticity.

### MEANS TO SOLVE THE PROBLEM

In order to attain the aforementioned object, the present inventors intensively studied and, as a result, it was made clear that, by adopting condition of a high concentration of 20 W/V % or higher of hyaluronic acid in a mixture to be subjected to a crosslinking reaction, a crosslinked hyaluronic acid gel having excellent viscoelasticity is obtained even when an amount of a crosslinking agent to be added is decreased. When general hyaluronic acid is used, a hyaluronic acid aqueous solution having a high concentration such as 20 W/V % or higher exhibits the solid powder state or the very highly viscous gel state, which is hardly called solution state. That is, the present inventors found out that, by reacting hyaluronic acid under the extremely high concentration condition which is not usually used by a person skilled in the art, a crosslinked hyaluronic acid gel exhibiting excellent viscoelasticity can be easily prepared although a crosslinking rate is low, which resulted in completion of the present invention.

The method of producing cross-linking hyaluronic acid gel of the present invention is characterized in that a mixture containing 20 W/V % or more of hyaluronic acid, a crosslinking agent and water is stirred and mixed under acidic or alkaline condition. In addition, in the method of producing cross-linking hyaluronic acid gel, it is suitable that a storage modulus G′ (frequency 1 Hz) of the mixture before subjected to a crosslinking reaction is 15000 Pa or higher.

In addition, in the method of producing cross-linking hyaluronic acid gel, it is preferable that the mixture is stirred and mixed with a rotation/revolution mixer. In addition, in the method of producing cross-linking hyaluronic acid gel, it is preferable that a concentration of a crosslinking agent in the mixture is 0.02 to 1 W/V %. In addition, in the method of producing cross-linking hyaluronic acid gel, it is preferable that a concentration of a crosslinking agent in the mixture is 0.02 to 2 W/W % relative to hyaluronic acid. In addition, in the method of producing cross-linking hyaluronic acid gel, it is preferable that a crosslinking agent is selected from the group consisting of divinylsulfone, 1,4-butanediol diglycidyl ether, and ethylene glycol diglycidyl ether.

### EFFECT OF THE INVENTION

According to the method of producing cross-linking hyaluronic acid gel of the present invention, a crosslinked hyaluronic acid gel having excellent viscoelasticity can be simply prepared although the crosslinking rate is low, by adopting condition of a high concentration of 20 W/V % or higher of hyaluronic acid in a mixture to be subjected to a crosslinking reaction.

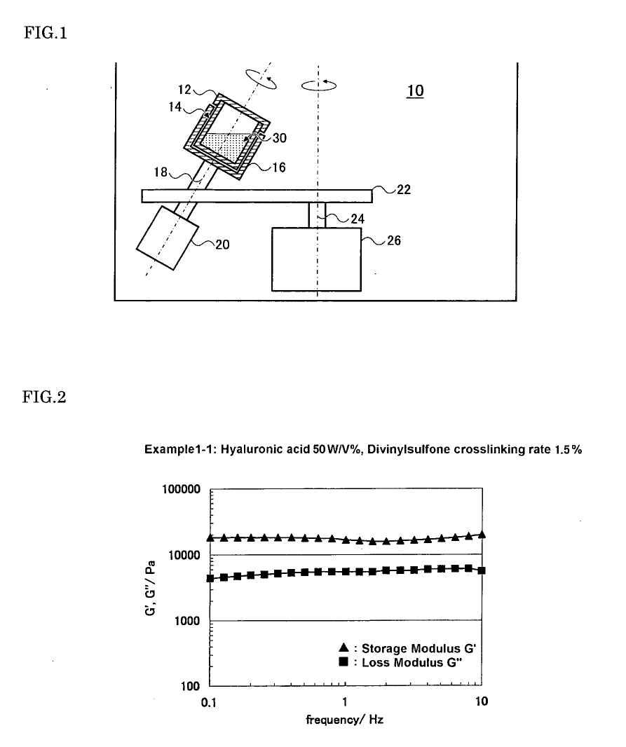

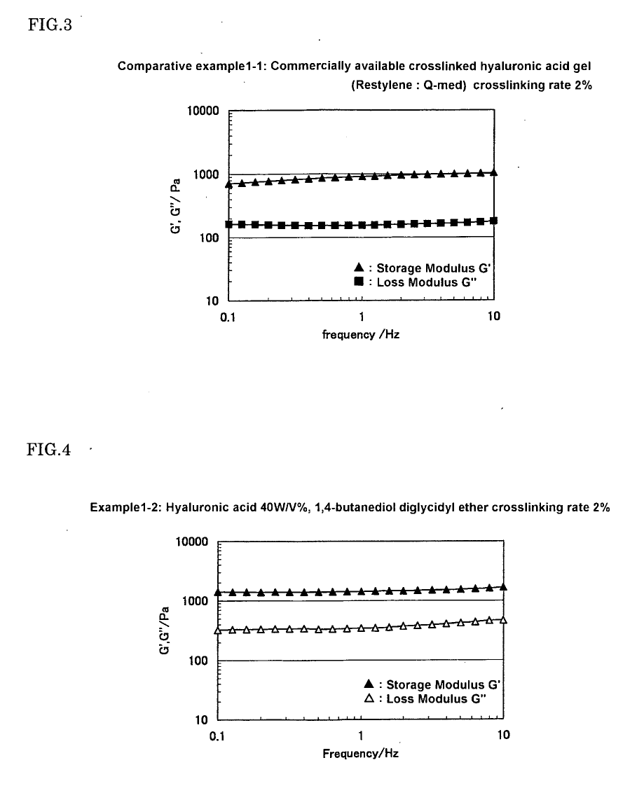

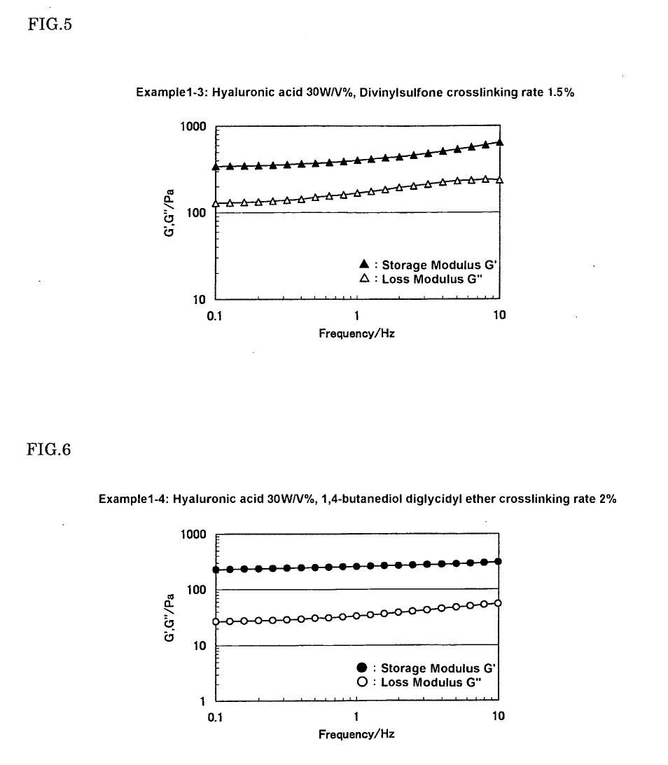

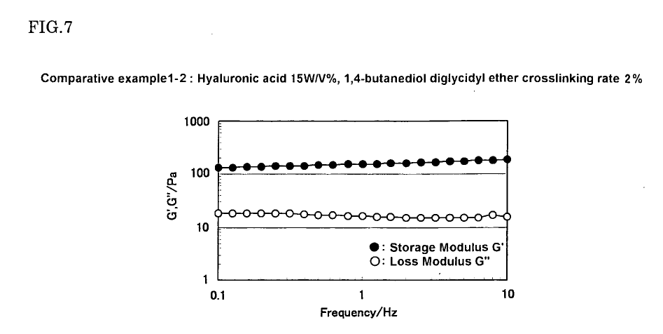

### BRIEF DESCRIPTION OF THE DRAWINGS

[FIG. 1] It is a view of an entire rotation/revolution mixer used in one example of the present invention.

[FIG. 2] It shows results of measurement of viscoelasticity of a crosslinked hyaluronic acid gel (hyaluronic acid 50 W/V %, divinylsulfone crosslinking rate 1.5%) obtained by Example 1-1 of the present invention.

[FIG. 3] It shows results of measurement of viscoelasticity of a commercially available crosslinked hyaluronic acid gel (Restylane: manufactured by Q-MED).

[FIG. 4] It shows results of measurement of viscoelasticity of a crosslinked hyaluronic acid gel (hyaluronic acid 40 W/V %, 1,4-butanediol diglycidyl ether crosslinking rate 2%) obtained in Example 1-2 of the present invention.

[FIG. 5] It shows results of measurement of viscoelasticity of a crosslinked hyaluronic acid gel (hyaluronic acid 30 W/V %, divinylsulfone crosslinking rate 1.5%) obtained in Example 1-3 of the present invention.

[FIG. 6] It shows results of measurement of viscoelasticity of a crosslinked hyaluronic acid gel (hyaluronic acid 30 W/V %, 1,4-butanediol diglycidyl ether crosslinking rate 2%) obtained in Example 1-4 of the present invention.

[FIG. 7] It shows results of measurement of viscoelasticity of a crosslinked hyaluronic acid gel (hyaluronic acid 15 W/V %, 1,4-butanediol diglycidyl ether crosslinking rate 2%) obtained in Comparative Example 1-2 of the present invention.

### BEST MODE FOR CARRYING OUT THE INVENTION

Embodiments of the present invention will be explained in detail below, but the present invention is not limited by them.

The method of producing cross-linking hyaluronic acid gel of the present invention is characterized in that a mixture containing 20 W/V % or more of hyaluronic acid, a crosslinking agent and water is stirred and mixed under acidic or alkaline condition. Thereby, a crosslinked hyaluronic acid gel having excellent viscoelasticity can be simply prepared although the crosslinking rate is low.

Hyaluronic acid used in the present invention is a straight-chain polymer in which a N-acetyl-D-glucosamine residue and a D-glucuronic acid residue are bound alternately as shown by the following general formula, and as far as hyaluronic acid has such a composition, it can be used without any limitation.

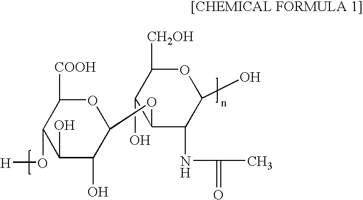

Hyaluronic acid can be obtained, for example, by isolation and extraction from a chicken crest or other animal tissue, or a fermentation method using a microorganism such as genus Streptococcus. In addition, in the present invention, for example, as a derivative of hyaluronic acid, a hyaluronic acid metal salt such as a sodium hyaluronate salt, a potassium hyaluronate salt and the like, or a hyaluronic acid derivative obtained by etherifying, esterifying, amidating, acetalizing, or ketalizing a hydroxyl group, a carboxyl group or the like of hyaluronic acid can be used.

Alternatively, as hyaluronic acid in the present invention, a commercially available product may be used. Examples of commercially available hyaluronic acid include Biohyalo 12 (manufactured by Shiseido), hyaluronic acid (manufactured by Kibun) and the like.

In the present invention, it is necessary that a mixture to be subjected to a crosslinking reaction contains the hyaluronic acid at 20 W/V % or more. In a mixture containing hyaluronic acid at a high concentration like this, since hyaluronic acid molecular chains are present in the state where they are entangled very complicatedly, molecular chains sterically restrain each other by partial crosslinking of hyaluronic acid chains, and a network structure can be stabilized firm. And, for this reason, it is considered that, by using a mixture containing 20 W/V % or more of hyaluronic acid in a crosslinking reaction, a crosslinked hyaluronic acid gel exhibiting excellent viscoelasticity can be prepared although a crosslinking rate is low. On the other hand, in the case where a hyaluronic acid concentration is lower than 20 W/V %, when a crosslinking rate is low, a freedom degree of a hyaluronic acid molecular chain is high and, as a result, a crosslinked gel becomes soft, and excellent viscoelasticity is not obtained. In addition, in the present invention, it is preferable that a mixture to be subjected to a crosslinking reaction contains 30 W/V % or more of the hyaluronic acid.

Usually, when generally used hyaluronic acid having a molecular weight of 100 thousands or higher is used to prepare a hyaluronic acid aqueous solution having a high concentration of the aforementioned 20 W/V % or more, the solid powder state or the very highly viscous gel state is exhibited, and a storage modulus G′ at a frequency of 1 Hz is 15000 Pa or higher. In the present invention, it is preferable that a reaction is performed in the state where a mixture to be subjected to a crosslinking reaction exhibits the solid powder state or the highly viscous gel state. That is, in the present invention, it is necessary that a crosslinking reaction is performed in the state where the hyaluronic acid molecules are entangled very complicatedly and, even if a hyaluronic acid concentration is 20 W/V % or higher, when a reaction is performed in the solution state where hyaluronic acid is dispersed in water, a crosslinked hyaluronic acid gel having desired viscoelasticity is not obtained in some cases. For this reason, in the present invention, it is preferable that a storage modulus G′ (frequency 1 Hz) of a mixture to be subjected to a crosslinking reaction is 15000 Pa or higher.

A molecular weight of hyaluronic acid used in the present invention is not particularly limited, but a molecular weight of 100 thousands or higher, further around 500 thousands to 3 million is preferable. Usually, generally used hyaluronic acid has a molecular weight of 100 thousands or higher in almost cases, but hyaluronic acid having a molecular weight of around 10 thousands, a molecular weight of which has been specially reduced, is also present. In the present invention, when this low-molecular hyaluronic acid having a molecular weight of around 10 thousands is used, even if a hyaluronic acid concentration is 20 W/V % or higher, hyaluronic acid is uniformly dispersed in water into the solution state in some cases and, when a crosslinking rate is low, a gel becomes soft, and desired viscoelasticity is not obtained in some cases, being not preferable.

As a crosslinking agent used in the present invention, any crosslinking agent may be used as far as it can crosslink between polymer chains of the aforementioned hyaluronic acid by a chemical bond. As a crosslinking agent for hyaluronic acid, a polyfunctional compound having two or more functional groups which can react with a reactive functional group in a hyaluronic acid molecule, such as a carboxyl group, a hydroxyl group and an acetamide group, to form a covalent bond can be used. Examples of a crosslinking agent used in the present invention include alkyldiepoxy bodies such as 1,3-butadiene diepoxide, 1,2,7,8-diepoxyoctane, 1,5-hexadiene diepoxide and the like, diglycidyl ether bodies such as ethylene glycol diglycidyl ether, 1,4-butanediol diglycidyl ether, bisphenol A diglycidyl ether and the like, divinylsulfone, epichlorohydrin. Among them, particularly, divinylsulfone, 1,4-butanediol diglycidyl ether, and ethylene glycol diglycidyl ether can be suitably used. In the present invention, two or more kinds of crosslinking agents may be used by appropriately combining them.

In the present invention, an amount of the crosslinking agent to be blended in a mixture which is subjected to a crosslinking reaction is not particularly limited, but from a viewpoint of biocompatibility of the resulting crosslinked hyaluronic acid gel, it is preferable to perform a crosslinking reaction with an as small amount as possible of a crosslinking agent. Specifically, a concentration of the crosslinking agent in a mixture to be subjected to a crosslinking reaction is preferably 0.02 to 1 W/V %, more preferably 0.05 to 0.5 W/V %. Alternatively, it is preferable that a concentration of a crosslinking agent in a mixture is 0.02 to 2 W/W % relative to hyaluronic acid. According to the present invention, even when a crosslinking agent concentration in a mixture is 1 W/V % or lower, a crosslinked hyaluronic acid gel having excellent viscoelasticity can be prepared. In the present invention, when a general low-molecular crosslinking agent is used, and a crosslinking reaction is performed using a crosslinking agent concentration of 1 W/V %, and a hyaluronic acid concentration of 20 W/V %, under presumption that a total amount of a crosslinking agent is reacted with a reactive group of hyaluronic acid, a crosslinking rate of the resulting crosslinked hyaluronic acid gel is usually about 15% or lower per one unit of a hyaluronic acid disaccharide repeating unit.

In the method of producing cross-linking hyaluronic acid gel of the present invention, by stirring and mixing a mixture containing 20 W/V % or more of the hyaluronic acid, the crosslinking agent and water under acidic or alkaline condition, to react a reactive functional group of the hyaluronic acid and the crosslinking agent, hyaluronic acid polymer chains are crosslinked by a chemical bond to produce a crosslinked hyaluronic acid gel.

In the present invention, for the purpose of enhancing reactivity of hyaluronic acid at a crosslinking reaction, a pH of a mixture is appropriately adjusted with an acid such as hydrochloric acid, sulfuric acid and the like, a base such as sodium hydroxide, potassium hydroxide and the like, or a suitable buffer such as a phosphate salt, a quaternary ammonium salt or the like, and stirring and mixing are performed under acidic or alkaline condition. Specifically, for example, it is preferable that a pH of a mixture is adjusted to 1 to 5 under acidic condition, or 10 to 14 under alkaline condition.

In addition, in the present invention, in addition to the aforementioned essential components, components which are usually used in medicaments, cosmetics or the like may be blended in a mixture to be subjected to a crosslinking reaction in advance, in such a range that the object and the effect of the present invention are not influenced. Examples of components which can be blended include ascorbic acid and a derivative thereof, a humectant such as glycerin and the like, retinol and a derivative thereof, and an anti-inflammatory agent such as salicylic acid and the like.

In the present invention, the method of stirring and mixing a mixture is not particularly limited, and it can be performed stirring and mixing by using known conventional mixing apparatuses. Specifically, the mixing apparatus used in the present invention is, for example, rotation/revolution mixer, three-roll mill, or bead mill type mixer and the like, but it is preferably used rotation/revolution mixer, since the operation is easy. In the case of using three-roll mill, it is difficult to operate in an aseptic condition, thus it is not preferable in view of application to living body. In addition, in the case of using bead mill type mixer, there is a problem that the separation of the bead from the obtained crosslinked gel is difficult.

In addition, upon preparation of a crosslinked hyaluronic acid gel, previously, a crosslinking reaction was generally performed in an around 0.1 to 3 W/V % hyaluronic acid aqueous solution. Hyaluronic acid as extremely high water swelling property and, for example, an about 10 W/V % hyaluronic acid aqueous solution exhibits the extremely highly viscous gel state. For this reason, for example, in the case of a general mixing apparatus such as a propeller mixer and a disper, it is difficult to uniformly stir and mix an entire system. Like this, since it was very difficult to perform a uniform crosslinking reaction in a hyaluronic acid aqueous solution under the high concentration condition.

To the contrary, in the method of producing cross-linking hyaluronic acid gel of the present invention, by performing stirring and mixing using a rotation/revolution mixer, it becomes possible to easily form a uniform crosslinked structure even under the extremely high hyaluronic acid concentration condition such as 20 W/V % or higher. Therefore, in the method of producing cross-linking hyaluronic acid gel of the present invention, it is preferably performed stirring and mixing using a rotation/revolution mixer.

The rotation/revolution mixer used in the present invention is known to a person skilled in the art and, for example, mixing apparatuses described in JP-A No.61-290946, JP-B No.5-32110, JP-A No.10-43568, JP-A No.11-226376, and JP-A No.2000-271465 can be used in the present invention.

The present invention will be explained in more detail below by way of embodiments of the method of producing cross-linking hyaluronic acid gel of the present invention.

A rotation/revolution mixer relating to one example of the present invention is shown in FIG. 1.

A rotation/revolution mixer 10 relating to one example of the present invention is provided with a container body 14 for accommodating a sample, and a lid 12 of the container, a container holder 16 for securing and retaining the container body 14, a rotation mechanism 20 which rotates the container holder 16 along a rotation axis 18, a supporting part 22 for supporting the rotation mechanism 20, and a revolution mechanism 26 for rotating the supporting part 22 along a revolution axis 24.

### PREPARATION EXAMPLE

1) A mixture 30 containing 20 W/V % or more of hyaluronic acid, a crosslinking agent and water is accommodated in a container body 14 of a rotation/revolution mixer 10, a container lid 12 is fitted, and this is mounted on a container holder 16.

2) The rotation/revolution mixer 10 is operated to rotate a rotation mechanism 20 and a revolution mechanism 26.

The mixture 30 contains hyaluronic acid at an extremely high concentration of 20 W/V % or higher, and exhibits the solid powder state or the very highly viscous gel state.

In the aforementioned Preparation Example, the container holder 16 is rotated by the rotation mechanism 20 along a rotation axis 18 and, further, a supporting part 22 supporting the rotation mechanism 20 is rotated by the revolution mechanism 26 along a revolution axis 24. And, the mixture 30 accommodated in the container holder 16 is rotated along the rotation axis 18 and, at the same time, is revolved along the revolution axis 24.

As described above, the mixture 30 containing 20 W/V % or more of hyaluronic acid, a crosslinking agent and water is subjected to the rotation/revolution mixer 10 having the rotation mechanism 20 and the revolution mechanism 26, and stirring and mixing are performed by revolution while rotated. Thereby, although the mixture 30 is in the solid powder state or the very highly viscous gel state, since an entire system is uniformly stirred and mixed without physical cutting of a molecular chain of a hyaluronic acid polymer, it becomes possible to easily form a uniform crosslinking structure between hyaluronic acid molecules.

In addition, the container holder 16 can perform rotation and revolution simultaneously by the rotation mechanism 20 and the revolution mechanism 26, and a rotation/revolution rate can be freely set. A rotation/revolution rate of the container holder 16 is different depending on a nature and a volume of a prepared crosslinked hyaluronic acid gel, and a scale of the container holder 16 and the rotation/revolution mixer 10, and usually, a rotation rate is around 60 to 1000 rpm, and a revolution rate is around 300 to 3000 rpm.

In addition, a time for stirring and mixing the mixture 30 with the rotation/revolution mixer 10 is different depending on the various conditions such as a nature and a volume of a prepared crosslinked hyaluronic acid gel and a scale of an apparatus as in the aforementioned rotation rate, but is usually around 10 seconds to 30 minutes.

According to the method of producing cross-linking hyaluronic acid gel relating to the present invention explained above, specifically, although a crosslinking rate is a low crosslinking rate of 2% or lower per one unit of a hyaluronic acid disaccharide repeating unit, for example, it becomes possible to simply prepare a crosslinked hyaluronic acid gel having very excellent viscoelasticity in which a storage modulus G′ was 1000 Pa or higher, and a loss modulus G″ was 2000 Pa or higher at a frequency of 1 Hz of the crosslinked hyaluronic acid gel which has reached swelling equilibrium in a physiological saline.

A crosslinked hyaluronic acid gel obtained by the process of the present invention, alone or by blending in a suitable formulation, can be used in medicaments, cosmetics or the like. In addition, according to the process of the present invention, since a crosslinked hyaluronic acid gel exhibiting excellent viscoelasticity can be simply prepared although a crosslinking rate is low, the gel can be applied to substances which are applied in a living body, for example, can be suitably applied to tissue increasing substances such as antiwrincle injections, drug sustained release compositions, adhesion preventing agents, or bone repairing agents.

### EXAMPLE 1

The present invention will be explained in more detail below by way of Examples of the present invention, but the present invention is not limited to them.

The present inventors first tried a crosslinking reaction with a small amount of a crosslinking agent under extremely high hyaluronic acid concentration condition such as 50 W/V %, viscoelasticity of the resulting crosslinked hyaluronic acid gel was measured, and this was compared with viscoelasticity of a commercially available crosslinked hyaluronic acid gel.

### EXAMPLE 1-1

### Hyaluronic Acid 50 W/V %, Divinylsulfone Crosslinking Rate 1.5%

200 μL of a 2N NaOH aqueous solution, and 2 μL of divinylsulfone were added to 1800 μL of purified water and, further, 1.0 g of hyaluronic acid (Biohyalo 12: manufactured by Shiseido) was added. This mixture (storage modulus G′ at frequency 1 Hz: 300000 Pa) was stirred and mixed at room temperature for 5 minutes with a rotation/revolution mixer (AR-250: manufactured by THINKY). The product was crushed with a mortar, and this was allowed to stand in a physiological saline for one week until it reached swelling equilibrium, to obtain a desired crosslinked hyaluronic acid gel. A storage modulus G′ and a loss modulus G″ of the resulting crosslinked hyaluronic acid gel were measured using a rheometer (Rheolyst AR1000-N: manufactured by TA Instruments) under the condition of 25° C. and a frequency of 0.1 to 10 Hz. Under the presumption that a total amount of an added crosslinking agent reacted with a reactive group of hyaluronic acid, a crosslinking rate of the resulting crosslinked hyaluronic acid gel is 1.5% per one unit of a hyaluronic acid disaccharide repeating unit. The results of measurement are shown in FIG. 2.

### COMPARATIVE EXAMPLE 1-1

### Commercially Available Crosslinked Hyaluronic Acid Gel (Restylane: Manufactured by Q-MED) Crosslinking Rate 1%

For the purpose of comparison with the present invention, regarding a commercially available crosslinked hyaluronic acid gel (Restylane: manufactured by Q-MED) which has previously been used widely as an antiwrinkle injection, a storage modulus G′ and a loss modulus G″ were measured as in Example 1-1. This commercially available crosslinked hyaluronic acid gel was prepared by the method described in Japanese Patent No.3094074, and it is presumed that a crosslinking rate is 2% per one unit of a hyaluronic acid disaccharide repeating unit. The results of measurement are shown in FIG. 3.

As shown in FIG. 2, it was revealed that a crosslinked hyaluronic acid gel obtained in Example 1-1, although a crosslinking rate is low as 1.5%, exhibits excellent viscoelasticity of a storage modulus G′ of about 20000 Pa and a loss modulus G″ of about 5000 Pa in a range of a frequency 0.1 to 10 Hz when the gel reaches swelling equilibrium with a physiological saline. This is thought as follows: Since a hyaluronic acid concentration in a mixture to be subjected to a crosslinking reaction is remarkably high as 50 W/V %, only by crosslinking with a small amount of a crosslinking agent, hyaluronic acid molecular chains are sterically restrained, and a network structure is stabilized firm. On the other hand, as shown in FIG. 3, it was seen that, in a commercially available hyaluronic acid gel of Comparative Example 1-1 which has previously been used widely as an antiwrinkle injection, a crosslinking rate is around 2%, and a storage modulus G′ was about 1000 Pa, and a loss modulus G″ was about 200 Pa when the gel reaches swelling equilibrium with a physiological serine. From this, it is seen that the crosslinked hyaluronic acid gel obtained in Example 1-1 has excellent viscoelasticity to a sufficient degree of actual use as an antiwrinkle injection although a crosslinking rate is low.

In addition, although the commercially available crosslinked hyaluronic acid gel used as Comparative Example 1-1 has a low crosslinking rate, a process for preparing it is, as described in Japanese Patent No.3094074, via a two-stage crosslinking reaction step of mixing hyaluronic acid and a crosslinked agent in an aqueous solution to initiate a crosslinking reaction, adding water before occurrence of gelling to dilute a mixed solution, thereby, preventing progression of a crosslinking reaction once, and volatilizing water from this mixed solution to progress a crosslinking reaction again, and very complicated procedure is required. It is seen that, to the contrary, according to the process of Example 1-1, by performing a crosslinking reaction under the extremely high hyaluronic acid concentration condition of 50 W/V %, a crosslinked hyaluronic acid gel having a low crosslinking rate and exhibiting excellent viscoelasticity can be prepared more simply.

Subsequently, the present inventors tried the same test as that of the Example 1-1 by appropriately changing a kind of a crosslinking agent and a hyaluronic acid concentration condition to be subjected to a crosslinking reaction, and studied relationship with viscoelasticity of the resulting crosslinked hyaluronic acid gel.

### EXAMPLE 1-2

### 40 W/V % Hyaluronic Acid 1,4-butanediol diglycidyl Ether Crosslinking Rate 2%

To 2250 μL of purified water were added 250 μL of a 2N NaOH aqueous solution, and 5 μL of 1,4-butanediol glycidyl ether and, further, 1.0 g of hyaluronic acid (Biohyalo 12: manufactured by Shiseido) was added. This mixture (storage modulus G′ at frequency of 1 Hz: 100000 Pa) was stirred and mixed with a rotation/revolution mixer (AR250: manufactured by THINKY) at room temperature for 5 minutes, and this was allowed to stand at room temperature for 24 hours. The resulting gel was allowed to stand in a physiological saline for one week until it reached swelling equilibrium, to obtain a desired crosslinked hyaluronic acid gel. The resulting crosslinked hyaluronic acid gel was measured for a storage modulus G′ and a loss modulus G″ as in Example 1-1. Under the presumption that a total amount of the added crosslinking agent reacted with a reactive group of hyaluronic acid, a crosslinking rate of the resulting crosslinked hyaluronic acid gel is 2% per one unit of a hyaluronic acid disaccharide repeating unit. The results of measurement are shown in FIG. 4.

As shown in FIG. 4, although the crosslinked hyaluronic acid gel of Example 1-2 obtained under the condition of hyaluronic acid concentration 40 W/V % using 1,4-butanediol diglycidyl ether as a crosslinking agent has a low crosslinking rate of 2%, when the gel reaches swelling equilibrium with a physiological saline, a storage modulus G′ was about 1500 Pa, and a loss modulus G″ was about 300 to 400 Pa in a range of a frequency of 0.1 to 10 Hz. From this, it was seen that, even when a different crosslinking agent from that of Example 1-1 is used, by performing a crosslinking reaction under the hyaluronic acid high concentration condition, a crosslinked hyaluronic acid gel having a low crosslinking rate and exhibiting excellent viscoelasticity is obtained.

### EXAMPLE 1-3

### 30 W/V % Hyaluronic Acid, Divinylsulfone Crosslinking Rate 1.5%

To 3000 μL of purified water were added 333 μL of a 2N NaOH aqueous solution and 2 μL of divinylsulfone and, further, 1.0 g of hyaluronic acid (Biohyalo 9: manufactured by Shiseido) was added. This mixture (storage modulus G′ at frequency 1 Hz: 60000 Pa) was stirred and mixed with a rotation/revolution mixer (AR250: manufactured by THINKY) at room temperature for 5 minutes. The product was crushed with a mortar, and this was allowed to stand in a physiological saline for one week until it reached swelling equilibrium, to obtain a desired crosslinked hyaluronic acid gel. The resulting crosslinked hyaluronic acid gel was measured for a storage modulus G′ and a loss modulus G″ as in Example 1-1. Under presumption that a total amount of the added crosslinking agent reacted with a reactive group of hyaluronic acid, a crosslinking rate of the resulting crosslinked hyaluronic gel is 1.5% per one unit of a hyaluronic acid disaccharide repeating unit. The results of measurement are shown in FIG. 5.

### EXAMPLE 1-4

### 30 W/V % Hyaluronic Acid, 1,4-butanediol diglycidyl Ether Crosslinking Rate 2%

To 3000 μL of purified water were added 333 μL of a 2N NaOH aqueous solution, and 5 μL of 1,4-butanediol diglycidyl ether and, further, 1.0 g of hyaluronic acid (Biohyalo 12: manufactured by Shiseido) was added. This mixture (storage modulus G′ at frequency 1 Hz: 60000 Pa) was stirred and mixed with a rotation/revolution mixer (AR-250: manufactured by THINKY) at room temperature for 5 minutes and this was allowed to stand at room temperature for 24 hours. The resulting gel was allowed to stand in a physiological saline for one week until the gel reached swelling equilibrium, to obtain a desired crosslinked hyaluronic acid gel. The resulting crosslinked hyaluronic acid gel was measured for a storage modulus G′ and a loss modulus G″ as in Example 1-1. Under presumption that a total amount of the added crosslinking agent reacted with a reactive group of hyaluronic acid, a crosslinking rate of the resulting crosslinked hyaluronic acid gel is around 2% per one unit of a hyaluronic acid disaccharide repeating unit. Results of measurement are shown in FIG. 6.

As shown in FIG. 5, in the case of Example 1-3 using divinylsulfone as a crosslinking agent under the condition of a hyaluronic acid concentration of 30 W/V %, a crosslinking rate was around 1.5% and, when the gel reached swelling equilibrium with a physiological saline, a storage modulus G′ was about 300 to 700 Pa, and a loss modulus G″ was about 100 to 200 Pa. In addition, as shown in FIG. 6, in the case of Example 1-4 using 1,4-butanediol diglycidyl ether, a crosslinking rate was around 2% and, when the gel reached swelling equilibrium with a physiological saline, a storage modulus G′ was about 200 Pa, and a loss modulus G″ was about 30 to 50 Pa. From these results, it was revealed that, by performing a crosslinking reaction under the hyaluronic acid high concentration condition, a crosslinked hyaluronic acid gel having excellent viscoelasticity can be obtained although a crosslinking rate is low such as around 1.5 to 2%.

### COMPARATIVE EXAMPLE 1-2

### 15 W/V % Hyaluronic Acid, 1,4-butanediol diglycidyl Ether Crosslinking Rate 2%

To 2250 μL of purified water were added 250 μL of a 2N NaOH aqueous solution, and 5 μL of 1,4-butanediol glycidyl ether and, further, 0.375 g of hyaluronic acid (Biohyalo 12: manufactured by Shiseido) was added. This mixture (storage modulus G′ at frequency 1 Hz: 20000 Pa) was stirred and mixed with a rotation/revolution mixer (AR-250: manufactured by THINKY), and this was allowed to stand at room temperature for 24 hours. The resulting gel was allowed to stand in a physiological saline for one week until the gel reached swelling equilibrium, to obtain a desired crosslinked hyaluronic acid gel. The resulting crosslinked hyaluronic acid gel was measured for a storage modulus G′ and a loss modulus G″ as in Example 1-1. Under presumption that a total amount of the added crosslinking agent reacted with a reactive group of hyaluronic acid, a crosslinking rate of the resulting crosslinked hyaluronic acid gel is 2% per one unit of a hyaluronic acid disaccharide repeating unit. The results of measurement are shown in FIG. 7.

As shown in FIG. 7, in the crosslinked hyaluronic acid gel of Comparative Example 1-2 obtained under the condition of a hyaluronic acid concentration of 15 W/V % using 1,4-butanediol diglycidyl ether as a crosslinking agent, although this was obtained under similar condition to Example 1-2 except for the concentration of hyaluronic acid, when the gel reached swelling equilibrium with a physiological saline, a storage modulus G′ was about 150 Pa, and a loss modulus G″ was about 20 Pa, and this was inferior to the crosslinked hyaluronic gels by above Examples. From this, it is thought that, in the present invention, a crosslinked hyaluronic acid gel having low crosslinking rate and excellent viscoelasticity can be prepared by adopting condition of a concentration of 20 W/V % or higher of hyaluronic acid in a mixture to be subjected to a crosslinking reaction.

[Explanation of Marks]

10 Rotation/revolution mixer
12 Lid
14 Container body
16 Container holder
18 Rotation axis
20 Rotation mechanism
22 Supporting part
24 Revolution axis
26 Revolution mechanism
30 Mixture

1. A process for preparing a crosslinked hyaluronic acid gel, comprising stirring and mixing a mixture comprising 10 W/V % or more of hyaluronic acid, a crosslinking agent and water under acidic or alkaline conditions.

2. The process for preparing a crosslinked hyaluronic acid gel according to claim 1, wherein the storage modulus G′ (frequency 1 Hz) of the mixture before said mixture is subjected to a crosslinking reaction of 15,000 Pa or higher.

3. The process for preparing a crosslinked hyaluronic acid gel according to claim 1, wherein the mixture is stirred and mixed without physical cutting of a hyaluronic acid polymer chain in the mixture.

4. The process for preparing a crosslinked hyaluronic acid gel according to claim 3, wherein the mixture is stirred and mixed with a rotation/revolution mixer.

5. The process for preparing a crosslinked hyaluronic acid gel according to claim 3, wherein the mixture is stirred and mixed with a dough kneading machine or a rice-cake making machine.

6. The process for preparing a crosslinked hyaluronic acid gel according to claim 3, wherein the mixture is stirred and mixed by kneading with human hands.

7. The process for preparing a crosslinked hyaluronic acid gel according to claim 1, wherein the crosslinking agent concentration in the mixture is 0.02 to 1 W/V %.

8. The process for preparing a crosslinked hyaluronic acid gel according to claim 1, wherein the crosslinking agent concentration in the mixture is 0.02 to 2 W/W % relative to the quantity of hyaluronic acid disaccharide repeating units.

9. The process for preparing a crosslinked hyaluronic acid gel according to claim 1, wherein the crosslinking agent is selected from the group consisting of divinylsulfone, 1,4-butanediol diglycidyl ether, and ethylene glycol diglycidyl ether.

10. The process for preparing a crosslinked hyaluronic acid gel according to claim 1, wherein the storage modulus G′ (frequency 1 Hz) of the mixture before said mixture is subjected to a crosslinking reaction is 60,000 Pa or higher.

11. The process for preparing a crosslinked hyaluronic acid gel according to claim 1, comprising stirring and mixing a mixture containing 11 W/V % to 41 W/V % of hyaluronic acid.

12. The process for preparing a crosslinked hyaluronic acid gel according to claim 1, comprising stirring and mixing a mixture containing 11 W/V % to 18.2 W/V % of hyaluronic acid.

13. The process for preparing a crosslinked hyaluronic acid gel according to claim 1, comprising stirring and mixing a mixture containing 18.2 W/V % to 33 W/V % of hyaluronic acid.

14. The process for preparing a crosslinked hyaluronic acid gel according to claim 1, comprising stirring and mixing a mixture containing 33 W/V % to 41 W/V % of hyaluronic acid.

15. The process for preparing a crosslinked hyaluronic acid gel according to claim 1, wherein the molecular weight of the hyaluronic acid is at least about 100,000.

16. The process for preparing a crosslinked hyaluronic acid gel according to claim 1, wherein the molecular weight of the hyaluronic acid is at least about 500,000.

17. The process for preparing a crosslinked hyaluronic acid gel according to claim 1, wherein the molecular weight of the hyaluronic acid is from about 500,000 to about 3,000,000.

18. The process for preparing a crosslinked hyaluronic acid gel according to claim 1, wherein the crosslinking agent concentration in the mixture is 0.05 to 0.5 W/V %.

19. The process for preparing a crosslinked hyaluronic acid gel according to claim 1, wherein the crosslinking agent is selected from the group consisting of 1,3-butadiene diepoxide, 1,2,7,8-diepoxyoctane, 1,5-hexadiene diepoxide, and bisphenol A diglycidyl ether.

20. The process for preparing a crosslinked hyaluronic acid gel according to claim 5, wherein the mixture is stirred and mixed with a dough kneading machine or a rice-cake making machine at a rate of about 1000 to about 2000 RPM.
